(19) United States
(12) Patent Application Publication
(10) Pub. No.: US 2026/0305983 A1
(43) Pub. Date: Oct. 8, 2026

(54) IMAGE POSITIONING FOR DIAGNOSTIC IMAGES

(71) Applicant: Welch Allyn, Inc., Skaneateles Falls, NY (US)

(72) Inventor: David L. Kellner, Baldwinsville, NY (US)

(21) Appl. No.: 19/342,920

(22) Filed: Sep. 29, 2025

Related U.S. Application Data
(60) Provisional application No. 63/784,516, filed on Apr. 7, 2025.

(51) Int. Cl. A61B 5/11; A61B 5/00

(52) U.S. Cl. CPC A61B 5/1128; A61B 5/0077; A61B 5/7264; A61B 5/7435; A61B 5/746; A61B 2562/0219

(57) ABSTRACT

An example system is described herein for a medical device coupled with functionality to capture digital images and positioning information of the medical device while the device is being used for an examination of an anatomical structure of a patient. The positioning information may be captured using motion or proximity sensors, such as accelerometers, gyroscopes, or inertial measurement units (IMUs). The example system may include associated methods implemented to determine relative positions of the captured images, quality of the captured images, and an extent of coverage of the anatomical structure obtained by the images. The system may provide a visualization of the captured images that maintains the relative positions, and allows an operator to browse, annotate or share the images. The system may further provide the captured images and associated relative positions as input to an automated diagnosis system.

### RELATED APPLICATIONS

This application claims priority to, and the benefit of, U.S. Provisional Patent Application No. 63/784,516, entitled “IMAGE POSITIONING FOR DIAGNOSTIC IMAGES,” filed Apr. 7, 2025, the contents of which are incorporated herein by reference in their entirety.

### TECHNICAL FIELD

This application relates generally to diagnostic systems and more specifically to improved image-based diagnostic tools, and associated systems and methods, for presenting and viewing images of anatomical structures.

### BACKGROUND

In various medical fields, clinicians diagnose health and diseases of patients by operating vision-aided instruments to examine parts of a patient's body. These instruments may be analog or digital. By way of example, during eye examinations, ophthalmoscopes allow a clinician to examine the fundus, retina, lens, and other parts of the eye. During ear examinations, otoscopes allow a clinician to examine the ear canal and eardrum. During examination of skin lesions, dermatoscopes allow a clinician to examine the surface and subsurface of the skin. Ophthalmoscopes, otoscopes, dermatoscopes, and other such vision-aided instruments are compact, portable optical instruments which can be handheld or otherwise manually aided.

Typically, the field-of-view of such vision-aided instruments is quite narrow (e.g., 10-20 degrees). For example, an ophthalmoscope lens may gather or condense light exiting a patient's pupil, presenting an image covering a portion of the fundus which the clinician can view. During an instrument-based visual examination, the clinician may manually guide such vision-aided instruments, to view various portions of the patient's eye, ear, skin, or other anatomical structures. Some vision-aided instruments may allow capture of diagnostic images of the portions of the target anatomy being viewed by the clinician, for use in diagnosis of conditions of the target anatomy after the visual examination. However, the diagnostic images captured during the visual examination may be challenging to browse, review, or otherwise interpret because they may be a set of unsorted and/or unaligned images, comprising hundreds of images, which may be in temporal order based on time of capture, but lacking a spatial context. It may also be difficult to easily see how the images relate to each other because of the geometry of the anatomy observed and the angle of observation. For example, flat images of the hemispherical retina may not easily align along edges. Additionally, it may not be easy to determine which portion of the target anatomy is depicted in an image, other images of the set that are related to the same portion, other images that are adjacent to the portion, and the like. For example, it may not be possible to determine relative positions of features observed in the images.

Thus, there is a need to present the diagnostic images in a form that allows for convenient browsing and interpretation by a clinician diagnosing a condition based on the images, or use by a software-based diagnosis system, and allow use of the images in additional ways during and after clinical examinations to enable saving, sharing, and annotation of the images. The various examples of the present disclosure are directed toward overcoming one or more of the deficiencies noted above.

### SUMMARY

Various implementations of the present disclosure relate to a medical device coupled with functionality to capture digital images and positioning information of the medical device while the device is being used for an examination of an anatomical structure of a patient. The positioning information may be captured using motion or proximity sensors, such as accelerometers, gyroscopes, or inertial measurement units (IMUs). Analysis of the images and positioning information may be used to provide a visualization of the captured images that maintains relative positions between the images, and allows an operator to browse, annotate or share the images, or diagnose conditions of the anatomical structure. The images and positioning information may also be provided as input to automated diagnosis systems.

In an example of the present disclosure, a system includes a memory, an image capture device, a display, and a processor operably connected to the memory, the image capture device, and the display, and computer-executable instructions stored in the memory and executable by the processor. The instructions, when executed by the processor, cause the processor to perform operations comprising: causing the image capture device to capture, during a first time period: a plurality of images of an anatomical structure of a patient, the plurality of images including a first image captured at a first time, and a second image captured at a second time, receiving motion data indicating movement of the image capture device during the first time period, determining, based on the motion data, a first position of the first image relative to a second position of the second image, generating, based on the first position and the second position, a visualization including the first image and the second image, wherein the visualization represents the first image and the second image in a two-dimensional space, and providing, via the display, a graphical user interface displaying the visualization.

In another example of the present disclosure, a method includes receiving, from an image capture device, a plurality of images of the anatomical structure including a first image captured at a first time and a second image captured at a second time after the first time, receiving motion data indicating movement of the image capture device between the first time and the second time, determining, based on the motion data, a relative position of the second image with respect to a first position of the first image, generating, based on the first position and the relative position, a visualization including the first image and the second image, wherein the visualization represents the first image and the second image in a two-dimensional space, and providing, via a display screen, a graphical user interface displaying the visualization.

In still another example of the present disclosure, a medical screening device includes an image sensor configured to capture an image, a positioning sensor configured to capture motion of the medical screening device, a processor operably connected to the image sensor and the positioning sensor, and memory storing instructions. The instructions, when executed by the processor, cause the processor to cause the image sensor to capture, during a first time period a plurality of images of an anatomical structure of a patient including a first image captured at a first time and a second image captured at a second time after the first time, cause the positioning sensor to capture motion data indicating movement of the medical screening device during the first time period, determine, based on the motion data, a relative position of the second image relative to a first position of the first image, generate, based on the relative position and the first position, a visualization including the first image and the second image, wherein the visualization represents the first image and the second image in a two-dimensional space, and provide, via a display screen associated with the medical screening device, a graphical user interface displaying the visualization.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates a system architecture and components for capturing diagnostic images and associated metadata using a vision-aided medical screening device.

FIGS. 2A-2D illustrate examples of medical screening devices, according to examples of the present disclosure.

FIG. 3 provides an illustration of a diagnostic display tool presenting a visualization of the diagnostic images, according to examples of the present disclosure.

FIG. 4 provides a first flow diagram illustrating an example method of the present disclosure.

FIG. 5 provides a second flow diagram illustrating an example method of the present disclosure.

In the figures, the left-most digit(s) of a reference number identifies the figure in which the reference number first appears. The use of the same reference numbers in different figures indicates similar or identical items or features. The drawings are not to scale.

### DETAILED DESCRIPTION

The present disclosure is directed, in part, to providing digital imaging solutions to accompany medical screening devices such as ophthalmoscopes, otoscopes, dermatoscopes, endoscopes, colposcopes, and the like. In some examples, the medical screening devices are handheld by a clinician and manually operated by moving the device to view various portions of a target anatomy of a patient such as an eye, an ear, a patch of skin, and the like. An example screening device of the present disclosure may be configured to capture digital images and/or video at various points during an examination of the target anatomy performed by the clinician, along with metadata that includes position information of the device at a time of capture.

The systems and methods described herein may provide, based on analysis of the position information and other metadata related to the images and/or video, visualizations of the captured images for displaying to a clinician to assist the clinician in determining a diagnosis of a condition of the target anatomy depicted in the images. In some examples, the clinician determining the diagnosis may be different from an operator of the medical screening device who performs the examination. The systems and methods may also enable the clinician performing the examination to save, share, annotate, and interact with stored images captured during the examination. Further, the systems and methods described herein may provide guidance to the clinician performing the examination to improve quality of the captured images and ensure coverage of the target anatomy.

The systems, methods, and devices described herein provide improvements over existing handheld medical screening devices that capture images. As an example, existing systems may produce output images which may be in temporal order based on time of capture. Such output images, which may comprise hundreds of images, may be difficult to browse or interpret as they lack a spatial context. For example, a clinician viewing the images may not know if an image is of a region above, below, to the left or to the right of a previous image without the spatial context. As another example, synthesizing, via software, an aggregated view of the target anatomy based on the images may be difficult. The systems, methods, and devices described herein generate spatial context of the captured images by tracking the relative positions of the capture device, and such information may be used as input to software-based interpretation and diagnosis systems. The systems, methods, and devices described herein may also provide visualization(s) that use the spatial context to enable a clinician performing diagnosis to browse and interpret the images with ease.

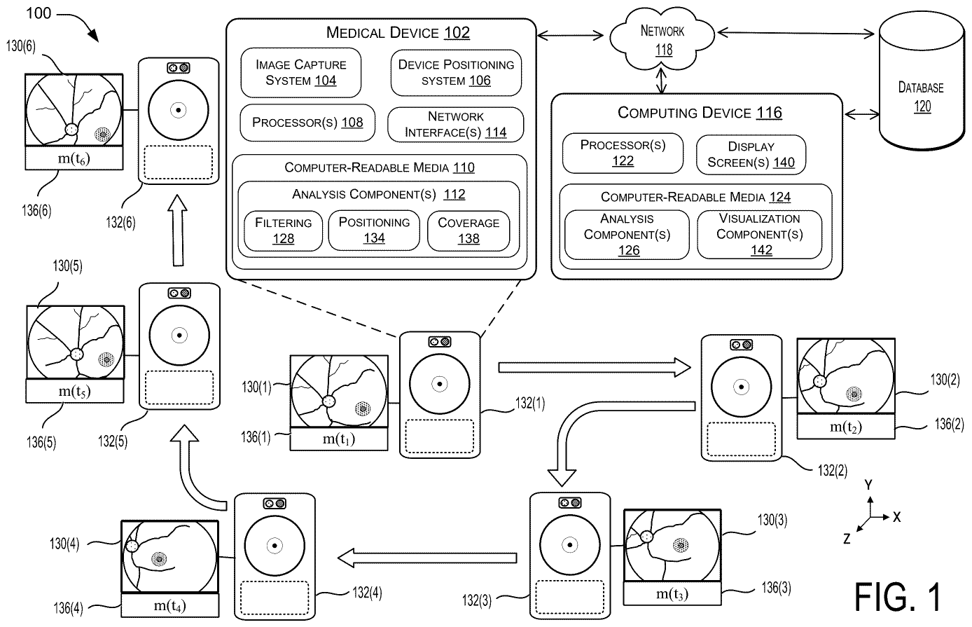

Details pertaining to the above-mentioned devices and techniques are described below with reference to FIGS. 1-5. It is to be appreciated that while these figures describe devices and systems that may utilize the claimed methods, the methods, processes, functions, operations, and/or techniques described herein may apply equally to other devices, systems, and the like.

FIG. 1 illustrates an example environment 100 for administering an examination using a medical device 102. The medical device 102 may be a portable or handheld device that may be positioned at different points to examine portions of a target anatomy of a patient by a clinician during the examination. As discussed above, some examples of the medical device 102 may include an ophthalmoscope, an otoscope, a dermatoscope, an endoscope, a colposcope, and the like. In examples, the medical device 102 may include an image capture system 104 and a device positioning system 106. In examples, the image capture system 104 may include a visible light camera (which may be full-color or greyscale), an infrared or near-infrared (NIR) camera, and/or one or more sensor(s) of other modalities. In some examples, the image capture system 104 may also include additional features such as various digital or optical filters (e.g., red-free, enhanced contrast, and the like), high-resolution cameras or high-speed video cameras, macro-lens, uniform or polarized lightning options, and the like.

In some examples, the image capture system 104 and/or the device positioning system 106 may be integrated into the medical device 102. In some examples, the image capture system 104 may only provide digital images and the medical device 102 may display digital images on a display screen of the device (not shown). In such examples, the clinician may view the target anatomy via the images on the display screen, and the image capture system 104 may save the digital images displayed on the screen for analysis and viewing after the examination. As another example, the image capture system 104 may be a digital camera that is attached to an analog viewer. For example, the medical device 102 may be an ophthalmoscope with an attached digital camera used for examining internal or external structures of the eye, an otoscope with an attached digital camera for imaging the ear canal and tympanic membrane of the ear, a dermatoscope with an attached digital camera, and the like.

In other examples, the image capture system 104 and/or the device positioning system 106 may be located on an external device functionally coupled with the medical device 102. For example, the medical device 102 may be an ophthalmoscope which is functionally and mechanically coupled with a smartphone that provides the capabilities of the image capture system 104 and the device positioning system 106. In some examples, the medical device 102 may correspond to handheld diagnostic tools which augment optical viewing with image capture, as described in U.S. patent application Ser. No. 18/959,333, filed on Nov. 25, 2024, the entire disclosure of which is incorporated herein by reference.

In some examples, the clinician operating the medical device 102 may, in the course of visually examining the target anatomy of the patient, concurrently operate a camera of the image capture system 104 to capture images of the target anatomy, such as an eye in the instance that the medical device 102 is an ophthalmoscope, an ear in the instance that the medical device 102 is an otoscope, skin in the instance that the medical device 102 is a dermatoscope, and the like. In some examples, the image capture system 104 may capture the images automatically, based on cues such as a pause in movement of the medical device 102, or identification of a target region of the anatomy. In some examples, the image capture system 104 may capture video images during a part or entirety of the examination, and/or the image capture system 104 may capture images at regular intervals (e.g., every 1/30 seconds, every 1/20 seconds, every 1/10 seconds, etc.). In some examples, the clinician may indicate a start of capture and an end of capture of images or video during the examination. In some examples, the clinician may indicate a series of start and end pairs, and the image capture system 104 may capture images in time intervals between a start and an end indication. Concurrently with the image capture system 104 capturing images or video, the device positioning system 106 may record position information of the medical device 102.

In some examples, the images may be captured through an additional optical instrument (such as a microscope, telescope, or borescope), or may depict any other view of the patient being examined. In some examples, the medical device 102 may include optical systems for visual examination by the clinician that are separate from optical systems of the image capture system 104. In other examples, the image capture system 104 may share some or all of the optical systems used for the visual examination. For example, the image capture system 104 may capture images of views of the target anatomy as seen by the clinician during the visual examination.

The device positioning system 106 may include position sensor(s) such as a proximity sensor, an accelerometer, a gyroscope and/or one or more other motion sensors. The position sensor outputs may be combined with a system clock output to indicate respective times corresponding to the position sensor outputs. In some examples, the accelerometer may comprise 6 degree-of-freedom (DOF) accelerometer-gyroscope combination that can track motion along 3 linear axes (X, Y, Z), and 3 rotational axes (yaw, pitch, roll). In other examples, the accelerometer may comprise a 3-axis linear accelerometer, and/or the gyroscope may track the 3 rotational axes of motion. Additionally or alternatively, the device positioning system 106 may use an inclinometer, a magnetometer, a MEMS inertial measurement unit (IMU), for tracking a position of the medical device 102. As known in the art, a positional displacement between two time instances may be computed from acceleration readings of the accelerometer by techniques such as double integration over a time period between the two time instances, using Kalman filter-based tracking of the motion, and the like.

Additionally or alternatively, the device positioning system 106 may utilize an image-based positioning system by tracking movement of pixels and features of images, such as using optical flow, in a video stream captured by the image capture system 104. The video stream may be captured at a high frame rate (e.g., high-speed video) to enable image-based motion tracking.

As shown schematically in FIG. 1, the medical device 102 may include processor(s) 108, which can be a single processing unit or a number of processing units, and can include single or multiple computing units or multiple processing cores. The processor(s) 108 can be implemented as one or more microprocessors, microcomputers, microcontrollers, digital signal processors, central processing units, state machines, logic circuitries, and/or any devices that manipulate signals based on operational instructions. For example, the processor(s) 108 can be one or more hardware processors and/or logic circuits of any suitable type specifically programmed or configured to execute the algorithms and processes described herein. In some examples, the processor(s) 108 may include graphics processing units (GPUs) to enable fast processing of image or video data.

In any of the examples described herein, the image capture system 104 and the device positioning system 106 may be operably connected to the processor(s) 108. In examples, the medical device 102 may also include computer-readable media 110 operably connected to the processor(s) 108. The processor(s) 108 can be configured to fetch and execute computer-readable instructions stored in the computer-readable media 110, which can program the processor(s) 108 to perform the functions described herein.

In examples, the computer-readable media 110 may be implemented using any suitable memory technology, such as static random-access memory (SRAM), synchronous dynamic RAM (SDRAM), double data rate SDRAM (DDR-SDRAM), nonvolatile/Flash-type memory, removable media such as an secure digital (SD) card, cloud-based memory, or any other type of memory capable of storing information. In some examples, the computer-readable media 110 may include dedicated RAM for handling video data, such as video RAM (VRAM).

The computer-readable media 110 can be used to store any number of functional components that are executable by the processor(s) 108. In examples, these functional components comprise instructions or programs that are executable by the processor(s) 108 and that, when executed, specifically configure the one or more processor(s) 108 to perform actions associated the examination performed with the medical device 102. Such actions may include image capture, recording image metadata associated with the capture, recording patient data associated with the patient being examined, and the like. For example, the computer-readable media 110 may store analysis component(s) 112, for analyzing data captured during the examination. In some examples, the processor(s) 108 and/or the computer-readable media 110 storing the analysis component(s) 112 may be included in a digital camera mounted to the medical device 102 as the image capture system 104. In some examples, the analysis of the captured data may be performed at a computing device separate from the medical device 102.

As an example, the medical device 102 may include network interface(s) 114 operably connected to one or more processor(s) 108. The medical device 102 may transmit the data captured during the examination to an external computing device 116, via a network 118. In some examples, the computing device 116 may be a computer proximate the medical device 102. For example, the computing device 116 may be located in an examination room where the examination is being performed. In some examples, the computing device 116 may be at a remote location or the computing device 116 may be implemented as a cloud service on a remote cloud server.

In some examples, the network interface(s) 114 may enable wired and/or wireless communications between the medical device 102 and one or more components of the computing device 116, as well as with one or more other remote systems and/or other networked devices. For instance, the network interface(s) 114 may communicate with a database 120 that may store patient information, including images captured during the examination of the patient and/or previous examinations of the patient. In some examples, the medical device 102 may store the captured data on the database 120 by transmitting such data via the network 118, and the computing device 116 may access the captured data from the database 120. In some examples, the database 120 may be a cloud-based storage or located on a server associated with a medical facility where the examination is performed.

In some examples, the network interface(s) 114 may include a personal area network component to enable communications over one or more short-range wireless communication channels, and/or a wide area network component to enable communication over a wide area network. In any of the examples described herein, the network interface(s) 114 may enable communication between, for example, the processor(s) 108 of the medical device 102, and processor(s) 122 of the computing device 116, via the network 118. The network 118 shown in FIG. 1 may be any type of wireless network or other communication network known in the art. Examples of network 118 include the Internet, an intranet, a wide area network (WAN), a local area network (LAN), and a virtual private network (VPN), cellular network connections and connections made using protocols such as 802.11a, b, g, n and/or ac.

In some examples, the medical device 102 may communicate via a wireless connection such as 802.11x, Bluetooth, Zigbee, or cellular network, with the computing device 116, which may be a smartphone, tablet, or computer in a same room (e.g., examination room) that the examination is performed. In some examples, the medical device 102 may communicate via a wireless connection or a wired ethernet connection with the computing devices 116 which may be a server within a medical facility where the examination is performed.

In alternative examples, the medical device 102 may store the data captured during the examination in a temporary storage (not shown), such as a flash drive, or other removable storage, and the like, on or associated with the medical device 102. Such data may then be transferred to the computing device 116 and/or the database 120 after completion of the examination. In such examples, the medical device 102 may not include the network interface(s) 114.

In examples, the computing device 116 may include computer-readable media 124 storing analysis component(s) 126, which may perform some or all of the functionalities of the analysis component(s) 112. For example, some analysis may be performed on the medical device 102 by the analysis component(s) 112 while other more computationally intensive analyses may be performed on the computing device 116 by the analysis component(s) 126. In some examples, outputs of the analysis performed by the analysis component(s) 112 or the analysis component(s) 126, as described below, may be stored on the database 120 and associated with the patient, so that the outputs can be accessed by other clinicians for diagnosis or other purposes.

In some examples, the computing device 116 may communicate, via the network 118, at least a portion of the outputs generated by the analysis component(s) 126 back to the processor(s) 108 of the medical device 102 while the examination is still in progress. In such examples, the portion of the outputs may be used to provide guidance to the clinician performing the examination. Alternatively or additionally, the portion of the outputs may be generated by the analysis component(s) 112 of the medical device 102. By way of example, the system described herein may provide, via the graphical user interface and in at least near real-time, a notification that, for example, a coverage level associated with images of anatomy is less than a threshold coverage level. In these examples, the notification may provide real-time feedback configured to guide a user of the image capture device. The notification may be associated with information other than the coverage level as well, and specifically may be associated with any other information described herein.

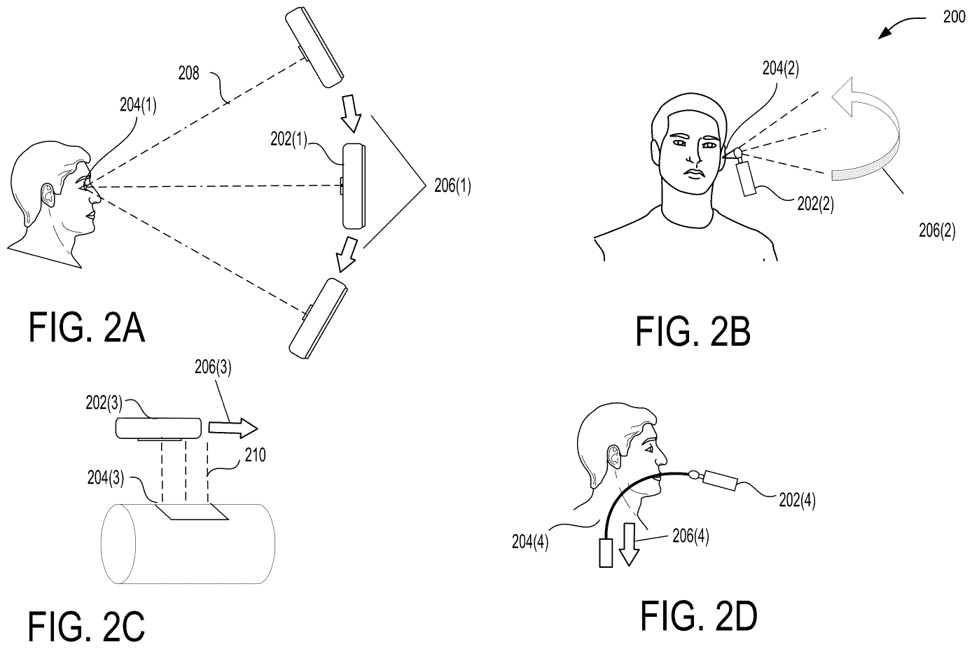

In examples, the medical device 102 may be moved to various positions during the examination to obtain different views of the target anatomy. In examples, the medical device 102 may have a narrow field-of-view (e.g., 20 degrees or less), and only a portion of the target anatomy may be visible from any given position. The positions the medical device 102 is moved to may vary based on a type of examination and the target anatomy as discussed further with reference to FIG. 2. In some examples, the medical device 102 may be a handheld device being operated by the clinician and may be moved manually. In some examples, the medical device 102 may be moved to the various positions by a robotic arm or electromechanical element programmed to move the medical device 102. In examples, movements of the medical device 102 may be small, due to constraints of the target anatomy and optical systems of the medical device 102.

In some examples, the image capture system 104 may capture image(s) while the examination is in progress, along with image metadata including time of capture. In examples, a filtering component 128 of the analysis component(s) 112 may take as inputs the captured image(s) to determine a set of images 130, which may be a subset of the captured image(s), that are suitable for visualization of the examination. Outputs of the filtering component 128 may include the set of images 130 of the captured images that satisfy quality criteria. In examples, the quality criteria may indicate that an image satisfying the quality criteria depicts the target anatomy, is of sufficient image quality, and/or anatomical structures needed for diagnostic use are visible in the image.

In some examples, at the start of the examination, the clinician may provide, via a user interface (not shown), an indication of a type of examination, such as an examination of the retina, cornea, and blood vessels of an eye, an eardrum or inner ear of an ear, a lesion on a patch of skin, and the like. In examples, the filtering component 128 may include machine learned (ML) model(s) or artificial intelligence system(s) trained using images of various target anatomies to identify whether an input image depicts the target anatomy of the type of examination. For example, an ML model may be trained on images of ear canals of patients, and such an ML model may be used to identify whether an input image depicts an ear canal. The filtering component 128 may provide, as input to the ML model(s), an image captured by the image capture system 104, and receive as an output from the ML model(s) whether the input image depicts the target anatomy expected for the type of examination. As an example, the ML model(s) may output a probability that the input image depicts the target anatomy, and the filtering component 128 may determine that the input image depicts the target anatomy if the probability is greater than a threshold probability.

Based on the output(s) of the ML model(s), the filtering component 128 may include, in the set of images 130, captured images that depict the target anatomy, and omit images of that do not depict the target anatomy. As examples, the removed images may include images captured before the target anatomy becomes visible during the examination, images captured accidentally, such as images of a floor, ceiling, a part of the patient that is not being examined, and the like, images captured while the medical device 102 is in motion where motion blur makes the target anatomy unclear, and the like. In some examples, the analysis component(s) 112 may start saving images captured by the image capture system 104 after a first time that the output of the filtering component 128 indicates that an input image depicts the target anatomy. For example, the first time may indicate that the target anatomy is acquired by the image capture system 104, and subsequent images are likely to represent small movements of the medical device 102 while keeping the target anatomy in its field-of-view.

In examples, the filtering component 128 may also include image processing, image recognition, and/or artificial intelligence algorithms to determine an image quality of an image such as sharpness, contrast, clarity, and the like. In examples, the filtering component 128 be configured to confirm an image quality of the images 130 captured by the image capture system 104. As used herein, the term “image quality,” and its equivalents, may refer to an extent to which an image clearly and accurately represents a subject or other item depicted in the image. Several factors may be associated with determining and/or characterizing image quality as being unacceptable, such as a blurriness of the image, under-or over-exposure, glare of light reflected from the target anatomy, or other distortions in the image. In some examples, if a quality of an image of the images 130 is determined to be below a threshold image quality, the filtering component 128 may omit the image from the set of images 130 output by the analysis component(s) 112. In some examples, a plurality of thresholds may be used, each characterizing a different factor, such as sharpness, exposure, presence of artifacts such as glare, and the like.

In some examples, the filtering component 128 may further analyze the images that are of a sufficient image quality (e.g., equal to or above the threshold image quality) to detect and identify anatomical structures visible within the images to determine if the anatomical structures required for diagnosis are visible, based on the type of examination selected. For example, an eye examination of the retina may require that an optic disc of the eye is visible in the image. In such an example, the filtering component 128 may use one more machine learned model(s) or image processing techniques to determine whether the optic disc is visible in the image. The filtering component 128 may only include images in the set of images 130 where the required anatomical structures are visible.

As illustrated in FIG. 1, the set of images 130 may include image(s) 130(1, . . . , n, . . . , N) captured from positions 132(1, . . . , n, . . . , N) respectively, at times t₁, t₂, . . . , tₙ, . . . , t_{N} respectively. Though an example with N=6 is illustrated, for a typical examination, N may be very large (e.g., hundreds).

In some examples, the device positioning system 106 may record the motion of the medical device 102, as motion data, throughout a duration of the examination, such as recording a time, and accelerometer and/or gyroscope readings at pre-determined intervals of 1/30 seconds or 1/60 seconds, and the like, from a starting time of the examination. In such examples, the motion data may comprise readings (e.g., every 1/30 seconds, 1/60 seconds, etc.) of the accelerometer and/or gyroscope and a corresponding time of capture of the readings.

In examples, the analysis component(s) 112 may also include a positioning component 134. The positioning component 134 may receive, as inputs, the motion data and an identification of the set of images 130. In examples, the positioning component 134 may determine spatial relationships between images of the set of images 130. In examples, the positioning component 134 may output position metadata 136(1, . . . , N) associated with each of the set of images 130, the position metadata 136 indicating the spatial relationships between images of the set of images 130.

In examples, the positioning component 134 may order the set of images 130 by time of capture (which is included in the respective image metadata) to determine an ordered set of images 130 (e.g., 130(1), 130(2), 130(3), 130(4), 130(5), 130(6) in the example shown). The position metadata 136(n) associated with a particular image 130(n), shown as m(tₙ) in FIG. 1, may be determined by using the respective time of capture, tₙ, of the image 130(n) to access the accelerometer and/or gyroscope readings recorded by the device positioning system 106 during a time duration from a starting time, t₀, of the examination up to time tₙ. In some examples, position metadata 136 may be relative to a previous image in the ordered set of images. For example, the position metadata 136(n) associated with the image 130(n) may be based on accelerometer and/or gyroscope readings between time tₙ and time tₙ₋₁, the capture time of a previous image 130(n−1). As an example, the position metadata 136(2) associated with the image 130(2) may be based on the motion data between time t₁ through time t₂. The positioning component 134 may store the position metadata 136 associated with the respective images 130(1, . . . , N) for further analysis to generate a set of spatially indexed images suitable for visualization of the examination. In some examples, the analysis component(s) 112 may provide the set of images 130 and associated position metadata 136 as input to an automated diagnosis system (not shown), thus providing information related to spatial relationships between images of the set of images to the automated diagnosis system.

In examples, the spatial relationships may be based on linear and/or rotational displacements in one or more linear or rotational axes, based on the type of examination being performed, as will be discussed further with reference to FIG. 2. As an example, the examination may involve imaging an anatomy that is substantially flat or having a small curvature, such as the retina of the eye, ear drum, skin patch, etc. In such examples, the image capture system 104 of the medical device 102 may also have a shallow depth of field which produces a sharp image of structures only within a small distance along an optical axis around a focal point where the medical device 102 is focused on. Accordingly, displacements along the optical axis of the image capture system 104, in a direction from the medical device 102 to the target anatomy, may be small or insignificant. As a result, the spatial relationship(s) determined by the positioning component 134 may be based on a two-dimensional plane, such as the X-Y plane shown, perpendicular to the optical axis of the medical device 102, which may be along the Z axis shown, and relative displacements between the images 130 in the third dimension, such as along the Z-axis, may be ignored. In other examples, the positioning component 134 may use readings from all three accelerometer axes, and determine a displacement in a three-dimensional space.

In some examples, the movements of the medical device 102 may be limited by constraints of the target anatomy. For example, within an ear canal, an otoscope may only be limited to rotational movement (e.g., rotate up to down, rotate left to right, etc.) after the otoscope reaches the tympanic membrane or eardrum, and the translational displacements along X, Y, Z axes may be small and can be ignored. In such examples, the positioning component 134 may represent the spatial relationship(s) as angular displacement between positions, determined based on rotational readings of the 6 DOF accelerometer or the gyroscope for viewing the tympanic membrane. In other examples, the movement of the medical device 102 may comprise translations along a single dimension only, such as moving along a linear path over a patch of skin. In such examples, the spatial relationship(s) determined by the positioning component 134 may simply use a displacement along a single axis along a direction of motion of the medical device 102 along the skin.

In some examples, the positioning component 134 may determine a relative displacement, which may be linear displacement and/or angular displacement, between an image 130(n) and a previous image 130(n−1) in the ordered set of images 130 based on the motion metadata. As an example, the positioning component 134 may determine a linear displacement between an image 130(n) and 130(n−1) by double integrating the accelerometer readings between time tₙ and tₙ₋₁. As discussed above, in some examples, the readings along the Z-dimension (as shown) may be ignored, and the linear displacement can be computed as a Euclidean distance in the X-Y plane. In other examples, the positioning component 134 may determine a rotational displacement between the image 130(n) and 130(n−1) by double integrating the readings of the rotational axes of a 6 DOF accelerometer or a gyroscope between the times tₙ and tₙ₋₁.

In some examples, the positioning component 134 may determine the position metadata 136 as displacement(s), such as along one or more of X, Y, Z, yaw, pitch, roll, of the images 130 with respect to an initial position. For example, the initial position may be the position 132(1) of the first image 130(1). In some examples, an image selected as corresponding to the initial position may be based on anatomical structure(s) and their position(s) depicted in the image. For example, for an eye examination, an image which depicts the optic disc at a particular position in the image may be selected as the initial position. In some examples, the positioning component 134 may determine the position metadata 136(n) corresponding to an image 130(n) as displacement(s) relative to the position 132(n−1) of the previous image 130(n−1), indicating the displacements along the X, Y, and/or Z-axis (or rotational displacement) from the position of the image 130(n−1).

In examples, the examination may follow a standard pattern of movements based on the type of examination. For example, during an eye examination, the medical device 102 may be an ophthalmoscope, and the clinician may move the ophthalmoscope to points along a circumference of a circle centered on the optical axis which may be aligned with a pupil of the eye. In some examples, the positioning component 134 may determine the position metadata 136 in terms of broad descriptors such as “left,” “right,” “above,” and “below,” to generally represent the typical movements and image captures followed during the examination. For example, the position metadata of the image 130(3) may be indicated as “below” and “right” of the image 130(2). In other examples, the medical device 102 may be an otoscope, and during the examination, a portion of the otoscope may be rotated within an ear canal to view different portions of the inner ear. In such examples, the position metadata may be expressed as a direction of rotation, such as “left,” “right”, “up”, “down”, and the like, and bins may indicate a difference in degrees of angle of rotation.

Additionally or alternatively, the positioning component 134 may also adjust a granularity of the position metadata 136. For example, the positioning component 134 may bin the displacements into ranges, rather than determine exact displacements. For example, the bins 1, 2, 3, . . . etc. may correspond to ranges 1:0-5 cm, 2:5-10 cm, 3:10-15 cm, etc. for linear displacements. As another example, the bins indicating rotational displacements may cover the ranges 1:0-5 degrees, 2:5-10 degrees, 3:10-15 degrees, etc. As an example, the positioning component 134 may determine the position metadata 136(3) of the image 130(3) relative to the image 130(2) as 1 unit “below” and 2 units to the “right.”

In some examples, the analysis component(s) 112 may include a coverage component 138 configured to determine whether views of the target anatomy depicted in the set of images 130 meet a coverage requirement that indicates that the set of images 130 provide views that are sufficient for diagnosis. For example, the coverage component 138 may access reference images associated with the type of examination, and use image matching techniques to determine matches between a reference image and one or more images of the set of images 130. In some examples, image matching may be based on features, such as edges, segments, or anatomical structures, present and location of the features in the two images being matched. For example, an optic disc of the eye may be detected in the set of images 130 and in the references images. For each position of the optic disc in a reference image, if the coverage component 138 detects one or more images of the set of images 130 where the optic disc is located within a threshold distance of the reference image, the coverage component 138 may determine that there is a match with the reference image. The coverage component 138 may output an indication that the coverage requirements are satisfied if the set of images 130 include each of the views depicted in the reference images. If matches are not found for one or more reference images, the coverage component 138 may output a set of reference images that did not have any match in the set of images 130.

In some examples, the coverage component 138 may access a list of positions from which images are to be captured for a given type of the examination. The coverage component 138 may determine whether at least one image of the set of images 130 was captured at each position in the list of positions, and determine if any position is missing a corresponding image. For example, an image may have been captured, but been filtered out because it failed to satisfy the quality criteria. The coverage component 138 may output a set of positions from which an image needs to be re-captured, or output an indication that the coverage requirements are satisfied.

In some examples, the coverage component 138 may be run while the examination is still in progress, such as after the images 130 have been captured, but before the patient is released. For example, the clinician may ensure that the images 130 satisfy the quality criteria and coverage needed for diagnosis, while there is an opportunity to re-capture images, if needed. In such examples, the coverage component 138 and the filtering component 128 may be run as components of the analysis component(s) 126 on the computing device 116, which may have more computing resources than the medical device 102, and outputs of the analysis component(s) 126 may be communicated to the medical device 102 over the network 118. In some examples, the analysis component(s) 126 may provide a notification to a clinician performing the examination, via display screen(s) 140 or by an interface of the medical device 102, if the outputs indicate a need for capturing additional image(s) or if the images captured are satisfactory. The notification may include information indicating coverage level and/or missing images or positions, allowing the clinician to capture images indicated.

In the environment 100 shown in FIG. 1, the display screen(s) 140 are shown as being associated with the computing device 116, which may be located in the examination room or may be remote. However, in other examples, the display screen(s) 140 may be associated with the processor(s) 108 of the medical device 102, and be configured to show output(s) from the analysis component(s) 112 of the medical device 102. In other examples, the computing device 116 may be a smartphone or a tablet and the display screen(s) 140 may comprise a screen of the smartphone or the tablet. In such examples, the image capture system 104 may be controlled via a mobile application (App) running on the smartphone or the tablet. For example, the clinician may provide the start of capture and the end of capture indications via the App. Additionally, the output(s) of the coverage component 138 may be displayed to the clinician during the examination via the App.

In some examples, the computer-readable media 124 of the computing device 116 may also store visualization component(s) 142 configured to process the set of images 130 and their corresponding position metadata 136 to generate a visualization of the set of images 130. Such a visualization may be used by the clinician performing the examination to verify that the set of images 130 provide sufficient information about the examination to allow for future diagnosis or comparisons. The visualization component(s) 142 may also be run on images and corresponding position metadata that are stored in the database 120 to generate the visualization. For example, the visualization may be generated at a later date from the examination, when a clinician accesses the set of images 130 to diagnose a condition of the patient. In examples, the visualization component(s) 142 may generate a two-dimensional (2D) representation where each image of the set of images 130 is placed at respective (x, y) coordinates in a two-dimensional space in accordance with the relative positions included in the position metadata 136. In some examples, the set of images 130 may be placed at respective (x, y, z) coordinates in a three-dimensional space, if the position metadata 136 indicates displacements along three axes.

In examples, the visualization component 142 may select a two-dimensional (2D) space and its dimensions (e.g., 4000×3000 pixels, 5000×5000 pixels, etc.). The visualization component 142 may then select an image of the set of images 130 as located at an origin (0, 0) of the 2D space. The selected image may be the first image 130(1) of the set of images 130, may be selected based on a location of an anatomical structure in the selected image (e.g., an image where the optic disc is centrally located in the image may be selected), or selected based on a central location among the positions 132. The visualization component 142 may map remaining images of the set of images 130 to (x, y) coordinates proportional to the relative displacements as indicated by the position metadata 136. For example, if the first image 130(1) is placed at an origin (0, 0) of the 2D space, the other images 130(2, . . . , 6) may be mapped to the 2D space based on their relative displacement with respect to the image 130(1). In examples, the visualization component 142 may select a mapping of one unit of displacement to n pixels based on a ratio between the maximum x and y values of the dimensions of the 2D space and the maximum x and y values of the displacements. For example, if the maximum displacement along the X axis is 10 cm, and the maximum x dimension is 4000, then each centimeter of displacement along the x-axis of the 2D space may be mapped to 400 pixels.

In the instance that the position metadata 136 comprises binned displacement values, the visualization component 142 may associate x and y coordinate values with each bin. For example, bin 1 may correspond to a distance along the x-axis or y-axis of 100 pixels, bin 2 may correspond to a distance along the x-axis or y-axis of 200 pixels, and the like. As an example, if image 130(2) is placed at position (x, y) in the 2D space, and the position metadata 136(3) indicates that image 130(3) is 1 unit “below” and 2 units to the “right” relative to the image 130(2), the image 130(3) may be placed at a position (x+400, y−100).

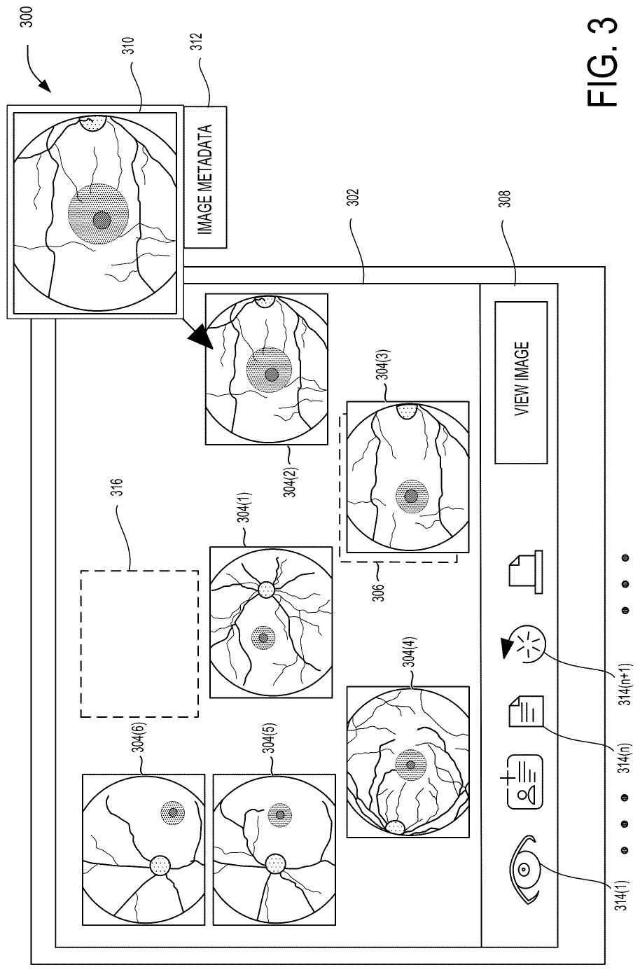

In some examples, the visualization component(s) 142 may additionally, or alternatively, generate a composite image or a mosaic by stitching the set of images 130 while maintaining the spatial relationships between the individual images. In some examples, such a composite image may comprise images stitched together, with overlapping portions removed, to generate a single image of the target anatomy. In some examples the visualization component(s) 142 may generate the composite image by incorporating central portions from the individual images of the set of images 130 while not including peripheral portions that are more likely to present distortions. In other examples, the visualization component(s) 142 may generate a visualization that includes complete images of the set of images 130 while maintaining their relative positions. In some examples, the images in such a visualization may overlap spatially, and the visualization may enable the clinician to browse side to side and up and down in a two-dimensional representation, as well as through a stack of overlapping images, as discussed in further detail with reference to FIG. 3.

In examples, the 2D representation generated by the visualization component(s) 142 may be provided via a user interface, along with tools for browsing, annotating, and sharing the set of images 130, as discussed in further detail with reference to FIG. 3. In some examples, the user interface may be displayed on the display screen(s) 140 associated with the computing device 116.

As discussed herein, FIG. 1 depicts an exemplary medical device 102 that includes components for capturing images and corresponding device positioning data at a time of capture of the images. The example system depicted may implement methods to determine relative positions of the captured images, quality of the captured images, and an extent of coverage of the target anatomy during an examination. The system may also provide a visualization, such as via a display screen associated with the medical device 102, of the captured images that maintains the relative positions, and enables a clinician, who may be different from the operator of the medical device 102, to easily review the captured images, such as for diagnosing a condition of the target anatomy. In some examples, one or more components may be implemented on a remote computing device 116 communicating with the medical device 102 over the network 118. The medical device 102 and its components are described in further detail with reference to the remaining figures.

FIG. 2A-2D illustrate various medical devices, which may correspond to the medical device 102 of FIG. 1, according to at least some examples. The medical devices depicted in FIGS. 2A-2D may be handheld or mounted devices that may be moved to obtain different views of a target anatomy. The medical devices depicted may be configured to capture digital images while being used for optical viewing by a clinician. In the examples shown, the medical devices 202(1), 202(2), 202(3), 202(4) may be used in examination of a target anatomy 204(1), 204(2), 204(3), 204(4) using various types of movements 206(1), 206(2), 206(3), 206(4). As discussed with reference to FIG. 1, the position metadata 136 may be based on the type of movements associated with the examination.

FIG. 2A illustrates an ophthalmoscope 202(1) for diagnosing conditions associated with an eye 204(1) of a patient. The ophthalmoscope 202(1) is designed primarily for performing diagnostic examinations of the eye of a patient and may be handheld by the clinician. The clinician may move the ophthalmoscope to various positions along a circumference of a circle centered on a pupil of the eye, to view internal structures of the eye through the pupil. The images captured from the various positions are essentially two-dimensional, as the curvature of the back of the eye is small. However, the edges of the images may be distorted and adjacent images may not be directly aligned. In examples, the ophthalmoscope 202(1) may have a shallow depth-of-field and therefore, to keep the retina in focus, the distance of the ophthalmoscope 202(1) may be held approximately same distance from the eye in the various positions. As a result, displacements caused by the movement 206(1) of the ophthalmoscope 202(1) between the various positions may be modeled in a two-dimensional plane perpendicular to an optical axis 208 of the ophthalmoscope 202(1).

FIG. 2B illustrates an otoscope 202(2) for diagnosing conditions of an ear 204(2) of a patient. The otoscope 202(2) is designed primarily for performing diagnostic examinations of the ear of a patient, although the device can also be used for examining other anatomical cavities (i.e., the nose, throat) of a patient. The otoscope 202(2) may also be handheld by the clinician, and may be partially inserted in the ear canal to examine the internal structures of the ear. Due to the anatomy of the ear canal, translational movements are limited after the otoscope 202(2) reaches the ear drum or tympanic membrane of the year. For example, the clinician may view structures or surfaces along the ear canal during the translational movements, but the movement 206(2) to view the tympanic membrane from different angles may be primarily rotational, where the clinician rotates the inserted portion of the otoscope 202(2) in the ear canal. In some examples, the displacements between the various positions of viewing the tympanic membrane may be modeled as primarily rotational, and expressed as angular displacements between the positions.

FIG. 2C illustrates a dermatoscope 202(3) for diagnosing conditions associated with a skin patch 204(3) of a patient. The dermatoscope 202(3) is designed primarily for performing diagnostic examinations of the skin of a patient, although the device can also be used for examining other anatomical structures of a patient. The dermatoscope 202(3) may also be configured to be handheld, but may also be mounted on a robotic arm to allow for smooth linear motion while scanning the skin patch associated with a condition. The dermatoscope 202(3) may be moved along a direction 206(3) parallel to the surface of the skin patch 204(3), while keeping a distance 210 over the skin surface 204(3) substantially fixed. In some examples, the displacements between images taken from various positions may be a linear distance along a direction of the movement 206(3) of the dermatoscope 202(3), and may be modeled as distances along a single axis. In other examples, the dermatosope 202(3) or the skin patch 204(3) (e.g., on an arm or leg) may be rotated to view the surface, and may be modeled as a rotational displacement along a rotational axis. In some examples, the movement 206(3) may be modeled as a combination of linear displacements and rotations.

FIG. 2D illustrates an endoscope 202(4) for viewing and diagnosing conditions associated with passages, and/or cavities of the body, such as upper gastrointestinal system or esophagus 202(4) shown. The endoscope 202(4) may be inserted by the clinician into an area to be examined, and images may be captured along a path of the endoscope. The movement 206(4) of the endoscope 202(4) may track along the anatomical passage or cavity being imaged. Such a movement 206(4) may cause a three-dimensional displacement between positions of the endoscope 202(4) e.g., along the X, Y and Z axes. In some examples, the rotational displacement may be limited due to constraints of the anatomy, and in such examples, the positions of the endoscope 202(4) may be modeled as three-dimensional displacements along X, Y, Z, without including rotational axes (yaw, pitch, roll).

FIG. 3 illustrates an example visualization 300 using an eye examination as an example of the type of examination. The visualization 300 illustrates images of the eye that may be captured by the medical device 102, such as the ophthalmoscope 202(1), and may aid a clinician or an operator of the vision screening device to diagnose conditions or diseases in the eyes of the patient. As described with reference to FIG. 1, the analysis component(s) 112 or 126 may process the captured images to determine the set of images 130 that satisfy quality criteria, and generate corresponding position metadata 136 associated with the set of images 130. Further, the visualization component 142 may generate a 2D representation of the set of images 130 providing (x, y) coordinates in a 2D space, or a 3D representation indicating (x, y, z) coordinates in a 3D space, associated with individual images of the set of images 130.

As shown in FIG. 3, using a 2D representation as example, the visualization 300 may be displayed on a display window 302, using thumbnail images 304(1, . . . , N) or downsampled versions of the set of images 130 at the coordinates indicated in the 2D representation. The display window 302 may display a portion of the 2D representation that fits the display window 302 at a given zoom level (which may be selectable by the operator). In examples, the display window 302 may be scrollable along X and Y axes to view the complete 2D space of the 2D representation (e.g., 4000×3000 pixels, 12000×8000 pixels, etc.) and/or provide interfaces to zoom out or zoom in to see more or less area of the 2D space. As illustrated in FIG. 3, the set of images 130, such as the images 130(1, . . . , 6) shown in FIG. 1, may be presented while maintaining their relative positions, allowing for ease of browsing and locating images that the operator or the clinician may want to inspect for determining the diagnosis. Though shown with spacing between the thumbnail images 304, at least some of the thumbnail images 304 may overlap and/or there may be multiple images at a same (x, y) coordinate or (x, y) coordinates in close proximity, which may be indicated as a stack of images 306. In examples, the stack of images 306 may show a substantially same portion of the target anatomy, which may be illuminated or captured from a viewing angle that is slightly different.

Though not shown, the display window 302 may also similarly display a three-dimensional (3D) representation, where the Z-axis may be perpendicular to a plane of the window 302. In such examples, the visualization 300 may provide 3D navigation tools to enable the clinician to browse the thumbnail images 304 in the 3D space. In some examples, the 3D representation may be mapped to a virtual 3D environment, enabling the clinician to browse the set of images 130 in their relative positions in the virtual 3D environment using devices such as a head-mounted virtual reality (VR) viewer. In some examples, the 3D representation may be converted or mapped to a 2D surface for visualization (e.g., using geo-mapping projections), and the 2D example visualization 300 shown in FIG. 3 may be used.

In examples, the visualization 300 may provide a panel of tools 308 to interact with the images 304 and the 2D representation (or a 3D representation) shown in the display window 302. In some examples, the panel of tools 308 may provide tools 314(1, . . . , n, . . . N) for viewing, annotating, and sharing the images 304. For example, a tool 314(n) may enable shuffling through the stack of images 306, bringing each image to the top for viewing. The panel of tools 308 may also include tools for annotating, saving and sharing one or more images representing by the thumbnail images 304. For example, the visualization 300 may enable an operator or the clinician may select a thumbnail, such as 304(2) shown, to view a larger, or full-resolution, version of the image 310 (of the set of images 130) represented by the thumbnail and/or image metadata 312 associated with the image 310. The visualization 300 may also enable the operator or the clinician to annotate the image 310, such as by adding highlights, circling portions, adding arrows, and the like, or add textual annotations describing features related to the diagnosis. Such annotations may be saved as image metadata 312 associated with the image 310.

In some examples, the panel of tools 308 may include a coverage tool, shown as 314(n+1), wherein, in response to selection of the coverage tool 314(n+1), the display window 302 may indicate area(s) 316 lacking coverage of the target anatomy, such as missing one or more reference images, as identified by the coverage component 138 discussed with respect to FIG. 1. The coverage tool 314(n+1) may be used during the examination to check whether more images need to be captured, such as in the area(s) 316 identified.

In examples, the operator or the clinician may simply hover over (e.g., via a mouse) or click on a thumbnail image, such as the thumbnail 304(2), in the display window 302 to access the full resolution image 310 and/or image metadata 312 associated with the image 310.

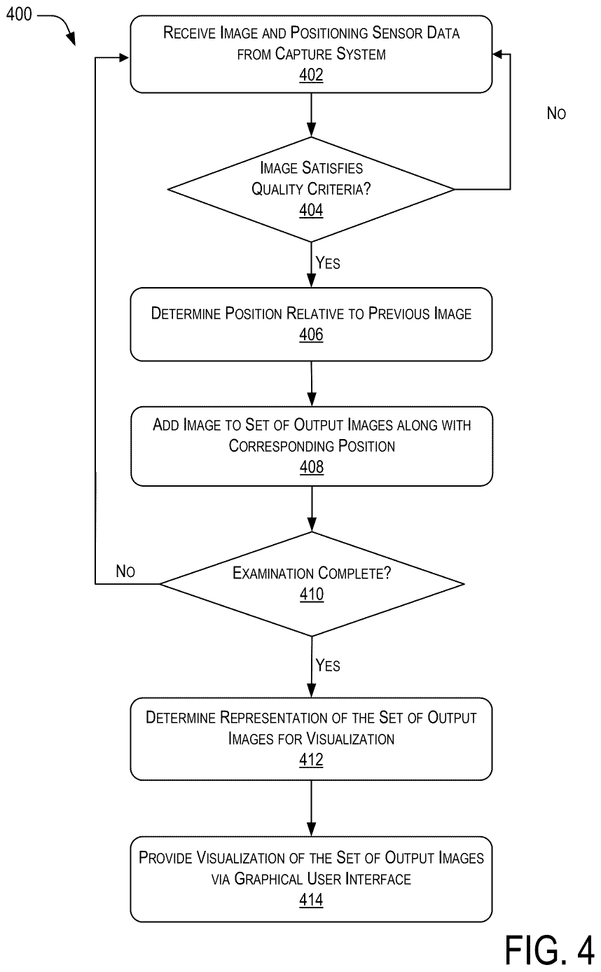

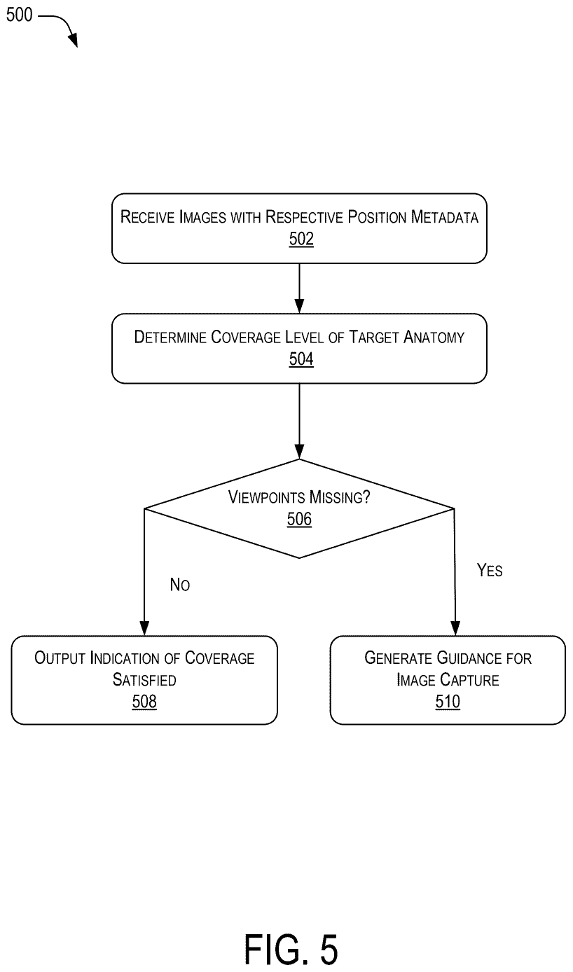

FIGS. 4 and 5 provide flow diagrams illustrating example methods for vision screening, as described herein. The methods in FIGS. 4 and 5 are illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the methods illustrated in FIGS. 4 and 5. In some embodiments, one or more blocks of the methods illustrated in FIG. 4 or 5 can be omitted entirely.

The operations described below with respect to the methods illustrated in FIGS. 4 and 5 can be performed by processors associated with any of the devices 102, 202(1-4), 116 described herein, and/or by various components thereof. Unless otherwise specified, and for ease of description, the methods illustrated in FIGS. 4 and 5 will be described below with reference to the environment 100 and the medical device 102 shown in FIG. 1. In particular, any of the operations described with respect to the methods illustrated in FIGS. 4 and 5 may be performed by the analysis component(s) 112, 126, or visualization component(s) 142 executed by the processor(s) 108, 122, either alone or in combination.

With reference to an example method 400 illustrated in FIG. 4, at operation 402, the processor(s) 108, 122 may receive image(s) captured by the image capture system 104 and positioning sensor data captured by accelerometer(s) and/or gyroscope(s) of the device positioning system 106 of the medical device 102. For example, the processor(s) 108, 122 may cause the image capture system 104 to capture the image(s) when an operator starts an examination by turning on the medical device 102 and/or entering a type of examination, which may indicate a target anatomy such as a retina of eye, an ear drum, a lesion on skin, and the like. The image(s) may also include image metadata such as time of capture. The positioning data may include accelerometer and/or gyroscope readings recorded at regular intervals e.g., 1/30 seconds or 1/60 seconds.

At operation 404, the processor(s) 108, 122 may, by executing the analysis component(s) 112 or 126, determine whether the image satisfies quality criteria, as described with reference to FIG. 1. For example, if the filtering component 128 determines that the image depicts the target anatomy, is of sufficient image quality, and/or anatomical structures needed for diagnostic use are visible in the image, the image may be determined to satisfy the quality criteria (Operation 404—Yes). In examples, some images may be captured by the image capture system 104 before the target anatomy becomes visible during the examination (e.g., the operator is still moving the medical device 102 to get the target anatomy in view), images may include those captured accidentally (e.g., of a floor, ceiling, a part of the patient that is not being examined, and the like), or images may be captured while the medical device 102 is in motion (e.g., where motion blur makes the target anatomy unclear). In such examples, the processor(s) 108, 122 may determine, as described with reference to FIG. 1, that the quality criteria are not satisfied (Operation 404—No), and further image(s) may be received at the operation 402.

If the processor(s) 108, 122 determines that the image satisfies the quality criteria (Operation 404—Yes), at an operation 406, the processor(s) 108, 122 may, by executing positioning component 134, determine a position associated with the image, which may be relative to a previous image. For example, the positioning component 134 may determine a spatial relationship between the image and a previous image based on motion metadata from the positioning sensors (e.g., accelerometers and/or gyroscopes) of the medical device 102. In some examples, the previous image may be the most recent image added to a set of output images maintained for the examination. In some examples, the position of subsequent images may be relative to the first image in the set. In yet other examples, the position may be determined relative to a reference position or reference image.

As described with reference to FIG. 1, the spatial relationship may be based on linear displacement(s) along x, y, and/or z-axis between the image and the previous image, and/or rotational displacement(s) (yaw, pitch, and/or roll) between the image and the previous image as determined from the positional sensor data. The axes (linear or rotational) along which displacement is determined may be based on constraints of the type of examination, as discussed with reference to FIG. 2. In some examples, the positioning component 134 may bin the displacements, or use broad categories indicating a direction of motion, such as “left,” “right,” “above,” or “below.”

At operation 408, the processor(s) 108, 122 may, by executing the analysis component(s) 112 or 126 may add the image to the set of output images for the examination, along with its corresponding position as determined at the operation 406. In some examples, the set of output images may be ordered by time of capture, and each image may be associated with position relative to the previous image.

At operation 410, the processor(s) 108, 122 may, by executing the analysis component(s) 112 or 126 may determine if the examination is complete. In some examples, the analysis component(s) 112 or 126 receive an indication that the examination is complete e.g., by the operator, using an interface of the medical device 102 such as an “end” button or by powering off. In some examples, the analysis component(s) 112 or 126 may determine that the examination is complete after not receiving additional images for a threshold period of time. If the examination is not complete (Operation 410—No), the analysis component(s) 112 or 126 may receive a next image at the operation 402.

If the examination is complete (Operation 410—Yes), at operation 412, the processor(s) 108, 122 may, by executing the visualization component(s) 142 may determine a representation of the set of output images for visualization. In some examples, the visualization may be a 2D representation that associated coordinates (x, y) with each image of the set of output images in a 2D space, that maintains the relative spatial relationships between images of the set of output images. For example, as described with reference to FIG. 1, the visualization component(s) 142 may select an image of the set of images 130 as located at an origin (0, 0) of the 2D space. The visualization component(s) 142 may then map remaining images of the set of output images to (x, y) coordinates in the 2D space that is proportional to the relative displacements as indicated by the position associated with the set of output images. For example, the visualization component(s) 142 may determine a ratio of the maximum displacement(s) with a maximum dimension of the 2D space, and scale the other displacement(s) according to the ratio.

In some examples, the displacement(s) may include 3D displacements along X, Y, and Z axes and/or rotational displacements (such as yaw, pitch, and roll). However, the displacements be small due to dimensions of the target anatomy. For example, the tympanic membrane in the ear may be 4-5 mm in diameter, the back of the eye may be 20-24 mm in diameter, and skin lesions may also be a few millimeters to a few centimeters in size. A shape of the surface being imaged may also be determined by the target anatomy being examined. For example, the back of the eye may present a surface shaped like an inside surface of a hemisphere, a surface of an arm with a skin lesion may be shaped like an outside surface of a cylinder, and the tympanic membrane may present an outside surface of a hemisphere. Based on known surface geometry of the target anatomy, knowledge of a distance of the image capture system 104 from the target anatomy, and knowledge of optical properties (e.g., focal length, field-of-view, lens characteristics, and the like) of the image capture system 104, the visualization component(s) may translate the observed displacement(s) to physical distances. Such physical distances may be mapped to a two-dimensional surface e.g., by projections of 3D points to 2D.

At operation 414, the processor(s) 108, 122 may, by executing the visualization component(s) 142 may provide a visualization, based on the 2D representation determined at the operation 412, for viewing via a graphical user interface (GUI), as described with reference to FIG. 3. As described, the visualization may provide a scrollable window over the 2D space, and thumbnail (or downsampled) images representing the set of output images may be placed at positions corresponding to their respective (x, y) coordinates in the 2D representation. The GUI may further provide tools for browsing, selecting, annotating, and sharing one or more images of the set of output images.

FIG. 5 illustrates an example method 500 for verifying that captured images during a medical examination provide the coverage of a target anatomy that is sufficient for diagnosing condition(s) associated with the target anatomy. In some examples, the method 500 may be executed online, such as while the examination is in progress, or shortly after the examination while the patient is still available for capturing additional images, if needed. One or more operations of the method 500 may be performed by the coverage component 138 executed by the processor(s) 108 or 122, as described with reference to FIG. 1.

At operation 502, the coverage component 138 may receive images with respective position metadata. For example, the images may correspond to the set of output images at a current time during the examination, such as obtained at the operation 408 of the method 400. The images may be captured by the medical device 102 during an examination of a particular type. In examples, the type of examination may provide information indicating a target anatomy and/or requirements for visibility of features of the target anatomy.

At operation 504, the processor(s) 108 or 122 may, by executing the coverage component 138, determine coverage level of the target anatomy based on the received images and their position metadata. As discussed with reference to FIG. 1, in some examples, the coverage component 138 may access reference images associated with the type of examination, and use image matching techniques to determine matches between a reference image and one or more images of the received images. In some examples, image matching may be based on features, such as edges, segments, or anatomical structures, present and location of the features in the two images being matched. Additionally or alternatively, the coverage component 138 may access a list of positions from which images are to be captured for the given type of the examination. The processor(s) 108 or 122 may determine whether at least one image of the received images was captured at each position in the list of positions, and determine if any position is missing a corresponding image. In examples, the coverage level may indicate a fraction or percentage of the reference images matched or a fraction or percentage of the positions in the list of positions that correspond to a captured image. In some examples, the processor(s) 108 or 122 may determine that a coverage level is sufficient if the coverage level meets or exceeds a threshold coverage level.

Further, in some examples, the coverage component 138 may access a list of anatomical structures required to be visible for the given type of examination. The coverage component 138 may use image processing technique and/or machine-learned models to determine whether at least one image of the received images depict each of the anatomical structures in the list. For example, if the list includes the optic disc of an eye, the coverage component 138 may use a machine-learned model trained to detect an optic disc in an image of the retina of an eye. The coverage component 138 may provide, as input, the received images to the trained machine-learned model to determine image(s) of the received images that include a depiction of the optic disc.

At operation 506, the coverage component 138 may determine whether viewpoints are missing, based on missing anatomical structures, missing matches to reference images, and/or missing views from reference list of positions for the type of examination. If the coverage component 138 determines that no viewpoints are missing (Operation 506—No), at operation 508, the coverage component 138 may output an indication that the coverage requirements are satisfied by the received images. The indication may be provided to the operator of the medical device 102 via an interface of the device, and may assist the operator in determining whether the examination can be ended, such as when the indication is received.

If at the operation 506, the processor(s) 108 or 122 determines, based on executing the coverage component 138, that viewpoints are missing (Operation 506—Yes), at operation 510, processor(s) 108 or 122 may generate guidance for image capture, which may be provided to the operator of the medical device 102 via an interface of the medical device 102. In examples, the guidance may comprise a list of missing viewpoints, which may be indicated by reference images that have no match in the received images, a list of missing positions from the list of positions associated with the type of examination, and/or an indication of anatomical structures that are not depicted in the received images. Such a guidance may allow the operator to further capture images from the missing viewpoints to complete the examination.

The foregoing is merely illustrative of the principles of this disclosure and various modifications can be made by those skilled in the art without departing from the scope of this disclosure. The above-described examples are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A system, comprising:
a memory;
an image capture device;
a display;
a processor operably connected to the memory, the image capture device, and the display; and
computer-executable instructions stored in the memory and executable by the processor to perform operations comprising:
causing the image capture device to capture, during a first time period a plurality of images of an anatomical structure of a patient, the plurality of images including a first image captured at a first time, and a second image captured at a second time;
receiving motion data indicating movement of the image capture device during the first time period;
determining, based on the motion data, a first position of the first image relative to a second position of the second image;
generating, based on the first position and the second position, a visualization including the first image and the second image, wherein the visualization represents the first image and the second image in a two-dimensional space; and
providing, via the display, a graphical user interface displaying the visualization.

2. The system of claim 1, wherein the second time is before the first time, and the operations further comprise determining, based on the first time and the second time, a subset of the motion data indicating movement of the image capture device between the first time and the second time, wherein the first position is determined based on the subset of the motion data.

3. The system of claim 1, wherein the motion data:
is captured by an accelerometer or gyroscope mechanically coupled to the image capture device; and
indicates a linear displacement or a rotational displacement.

4. The system of claim 1, the operations further comprising:
identifying, utilizing a trained machine learning model, a target anatomy depicted in a subset of the plurality of images;
determining that the subset of the plurality of images satisfies a quality criteria; and
determining a respective location of each image of the subset of the plurality of images in the two-dimensional space, wherein the visualization includes a representation of each image of the subset.

5. The system of claim 4, wherein an image satisfying the quality criteria is characterized by at least one of:
an image quality of the image exceeding a threshold image quality;
the image depicting the anatomical structure; or
the image depicting one or more anatomical features of the anatomical structure.

6. The system of claim 1, wherein the graphical user interface includes a scrollable window displaying the two-dimensional space, the two-dimensional space including:
a downsampled view of the second image at a first location; and
a downsampled view of the first image at a second location, the second location based on a distance between the first position and the second position, wherein the downsampled views are selectable to access a full-resolution view of the first image or the second image.

7. The system of claim 1, wherein the anatomical structure comprises one of an eye, an ear, or a skin surface.

8. The system of claim 1, the operations further comprising:
determining, based on the plurality of images, a coverage level of the anatomical structure, the coverage level indicating a proportion of the anatomical structure depicted in the plurality of images;
determining that the coverage level is less than a threshold coverage level; and
providing, via the graphical user interface and in at least near real-time, a notification that the coverage level is less than the threshold coverage level, wherein the notification provides real-time feedback configured to guide a user of the image capture device.

9. The system of claim 1, the operations further comprising:
determining, based on a set of reference views of the anatomical structure, one or more reference views that are omitted by the plurality of images; and
providing, via the graphical user interface, an indication of the one or more reference views.

10. The system of claim 9, the operations further comprising:
determining, based on the motion data and a set of reference positions of the anatomical structure, one or more reference positions that are omitted by the plurality of images; and
providing, via the graphical user interface, an indication of the one or more reference positions.

11. A method of generating a visualization of an anatomical structure, comprising:
receiving, from an image capture device, a plurality of images of the anatomical structure including a first image captured at a first time and a second image captured at a second time after the first time;
receiving motion data indicating movement of the image capture device between the first time and the second time;
determining, based on the motion data, a relative position of the second image with respect to a first position of the first image;
generating, based on the first position and the relative position, a visualization including the first image and the second image, wherein the visualization represents the first image and the second image in a two-dimensional space; and
providing, via a display screen, a graphical user interface displaying the visualization.

12. The method of claim 11, wherein the motion data comprises sensor data from an accelerometer or gyroscope coupled to the image capture device.

13. The method of claim 11, further comprising:
determining a subset of the plurality of images that satisfy a quality criteria; and
determining a respective location of each image of the subset of the plurality of images in the two-dimensional space, wherein the visualization includes a representation of each image of the subset.

14. The method of claim 13, wherein an image satisfying the quality criteria is indicative of:
an image quality of the image exceeding a threshold image quality;
the image depicting the anatomical structure; or
the image depicting one or more anatomical features of the anatomical structure.

15. The method of claim 11, wherein the motion data comprises one or more of:
a linear displacement along one, two, or three perpendicular axes;
a rotational displacement;
an indication of direction of motion; or
an indication of an amount of motion.

16. The method of claim 11, further comprising:
determining, based on the plurality of images, a coverage level of the anatomical structure the coverage level indicating a proportion of the anatomical structure depicted in the plurality of images;
determining that the coverage level is less than a threshold coverage level; and
providing, via the graphical user interface, a notification that the coverage level is less than the threshold coverage level.

17. A medical screening device, comprising:
an image sensor configured to capture an image;
a positioning sensor configured to capture motion of the medical screening device;
a processor operably connected to the image sensor and the positioning sensor; and
memory storing instructions that, when executed by the processor, cause the processor to:
cause the image sensor to capture, during a first time period a plurality of images of an anatomical structure of a patient including a first image captured at a first time and a second image captured at a second time after the first time;
cause the positioning sensor to capture motion data indicating movement of the medical screening device during the first time period;
determine, based on the motion data, a relative position of the second image relative to a first position of the first image;
generate, based on the relative position and the first position, a visualization including the first image and the second image, wherein the visualization represents the first image and the second image in a two-dimensional space; and
provide, via a display screen associated with the medical screening device, a graphical user interface displaying the visualization.

18. The medical screening device of claim 17, wherein the anatomical structure comprises one of an eye, an ear, or a surface of skin.

19. The medical screening device of claim 17, wherein instructions further cause the processor to:
determining, based on the plurality of images, a coverage level of the anatomical structure the coverage level indicating a proportion of the anatomical structure depicted in the plurality of images;
determining that the coverage level is less than a threshold coverage level; and
providing, via the graphical user interface, a notification that the coverage level is less than the threshold coverage level.

20. The medical screening device of claim 17, wherein providing the graphical user interface displaying the visualization comprises providing the graphical user interface via a virtual reality viewer configured to display the visualization in three-dimensions.
